(19) United States
(12) Patent Application Publication
(10) Pub. No.: US 2023/0363912 A1
(43) Pub. Date: Nov. 16, 2023

(54) INTERCOMMISSURAL LEAFLET SUPPORT

(71) Applicant: Edwards Lifesciences Innovation (Israel) Ltd., Caesarea (IL)

(72) Inventor: Yaron Herman, Givat Ada (IL)

(21) Appl. No.: 18/352,924

(22) Filed: Jul. 14, 2023

Related U.S. Application Data
(63) Continuation of application No. PCT/IB2022/050008, filed on Jan. 2, 2022.
(60) Provisional application No. 63/138,299, filed on Jan. 15, 2021.

(51) Int. Cl. A61F 2/24; A61L 27/36

(52) U.S. Cl. CPC A61F 2/2463; A61L 27/3625; A61L 27/367; A61L 2430/20

(57) ABSTRACT

An anchor is anchorable to tissue of a ventricle downstream of a heart valve of a subject. Each wing of a pair of wings defines a lateral surface and a medial surface, such that the medial surface of one wing of the pair faces the medial surface of the other wing of the pair. The wings are coupled to the anchor such that, when the anchor is anchored to the tissue, the anchor supports the wings at the valve, with the lateral surface of each wing facing a respective leaflet of the valve. During systole, the lateral surface of each wing is in contact with the respective leaflet, and the medial surfaces of the wings move into contact with each other, obstructing retrograde blood flow. During diastole, the medial surfaces move out of contact with each other, facilitating antegrade blood flow. Other implementations are also described.

### CROSS-REFERENCES TO RELATED APPLICATIONS

The present application is a Continuation of International Patent Application PCT/IB2022/050008 to Herman, filed Jan. 2, 2022, and titled “Intercommissural leaflet support,” which published as WO 2022/153131, and which claims priority to U.S. Provisional Patent Application 63/138,299 to Herman, filed Jan. 15, 2021, and titled “Intercommissural leaflet support.” Each of the above applications is incorporated herein by reference.

### BACKGROUND

Certain cardiac pathologies, such as dilation of the annulus of a heart valve, and chordae tendineae rupture, can prevent the valve leaflets from fully coapting when the valve is closed, and/or result in leaflet flail. Regurgitation of blood from the ventricle into the atrium can result in increased total stroke volume and decreased cardiac output, and ultimate weakening of the ventricle secondary to a volume overload and a pressure overload of the atrium.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

Some applications herein are directed to systems, apparatuses, and methods for use of a leaflet support to reduce regurgitation of a heart valve of a subject. The leaflet support can provide a substitute coaptation surface, and/or restrain the leaflets from flailing.

For some applications, a leaflet support is used to facilitate functioning of a valve of the heart. For some such applications, the leaflet support facilitates antegrade blood flow through the valve during diastole. For some such applications, the leaflet support obstructs retrograde blood flow through the valve during systole.

For some applications, the leaflet support comprises a pair of wings, each wing defining: (i) a medial surface that faces the medial surface of the other wing, and (ii) a lateral surface that faces away from the other wing. Often for such applications, the leaflet support is implanted (e.g., anchored to ventricular tissue) such that the lateral surface of each wing faces a respective leaflet of the valve.

Further for applications in which the leaflet support comprises a pair of wings, the leaflet support can be implanted to obstruct retrograde blood flow during systole by: (i) the medial surfaces of the wings contacting each other and (ii) the lateral surfaces of the wings contacting the leaflets.

Further for applications in which the leaflet support comprises a pair of wings, the leaflet support can be implanted to facilitate antegrade blood flow during diastole by the medial surfaces of the wings moving out of contact with each other.

For some applications, the lateral surfaces remain in contact with the leaflets also during diastole. For some such applications, the wings are affixed to the leaflets. For example, the wings can be stapled and/or adhered to the leaflets. Alternatively or in addition, the wings can comprise material that encourages ingrowth of tissue in contact with the lateral surfaces. For example, the wings can comprise an integrin-binding ligand.

For some applications, the leaflet support comprises a coaptation element and a fixator. Often for such applications, the fixator is placed behind a downstream surface of a leaflet of the valve, such that the fixator supports the coaptation element between a first leaflet and a second leaflet (e.g., underneath or on the ventricular side of the leaflet). In this way, the second leaflet: (i) coapts with the coaptation element during systole, and (ii) deflects relative to the coaptation element during diastole.

Further often for applications in which the leaflet support comprises a fixator, the fixator passively fills with blood after being implanted. For some such applications, fixator filling with blood facilitates securing fixator to the implantation site (e.g., by increasing a volume of the fixator). For example, the fixator can include an absorbent material and/or a shape-memory material.

For some such applications, the fixator filling with blood can facilitate coaptation of the second leaflet with the coaptation element during systole. For example, the fixator filling with blood may press the first leaflet against coaptation element, and/or may push the first leaflet toward the second leaflet.

There is therefore provided, in accordance with some applications, a system and/or an apparatus for use with a valve of a heart of a subject, the heart cycling between systole and diastole, and the system and/or apparatus including a leaflet support. For some applications, the leaflet support includes a tissue anchor, anchorable to ventricular tissue of a ventricle that is downstream of the valve and a pair of wings.

For some application, each wing of the pair of winds defines a medial surface, such that the medial surface of one wing of the pair faces the medial surface of the other wing of the pair, and a lateral surface.

For some applications, the pair of wings is coupled to the tissue anchor such that, when the tissue anchor is anchored to the ventricular tissue, the tissue anchor flexibly supports the pair of wings at the valve. For some applications, the pair of wings are configured and/or supported such that the lateral surface of each wing of the pair facing a respective leaflet of the valve such that, during systole, the lateral surface of each wing of the pair is in contact with the respective leaflet and the medial surfaces of the wings of the pair move into contact with each other, thereby obstructing retrograde blood flow through the valve. During diastole, the medial surfaces of the wings move out of contact with each other, thereby facilitating antegrade blood flow through the valve.

There is therefore provided, in accordance with some applications, a system and/or an apparatus for use with a valve of a heart of a subject, the heart cycling between systole and diastole, and the system and/or apparatus including a leaflet support. For some applications, the leaflet support includes a tissue anchor, anchorable to ventricular tissue of a ventricle that is downstream of the valve, and a wing. The wing has a lateral surface configured to face and contact a first leaflet, and an opposite medial surface configured to face a valve opening and a second leaflet.

For some applications, the wing is coupled to the tissue anchor such that, when the tissue anchor is anchored to the ventricular tissue, the tissue anchor flexibly supports the wing at the valve. For some applications, the wing is configured and/or supported such that, during systole, the lateral surface is in contact with the first leaflet and the medial surface is moved into contact with the second leaflet, thereby obstructing retrograde blood flow through the valve. During diastole, the medial surface moves out of contact with the second leaflet, thereby facilitating antegrade blood flow through the valve.

For some applications, the lateral surface includes an entire area of the wing.

For some applications, the leaflet support has a delivery state in which the leaflet support is configured to be transluminally advanceable to the valve.

For some applications, the pair of wings is a first pair of wings, and the leaflet support includes a second pair of wings. For some applications, the leaflet support includes a third pair of wings.

For some applications, each wing of the pair includes a material that is impermeable to blood.

For some applications, each wing of the pair includes pericardial tissue.

For some applications, the pair of wings is configured such that, when the tissue anchor is anchored to the ventricular tissue, each wing of the pair remains in contact with its respective leaflet during diastole.

For some applications, the pair of wings is configured such that, when the tissue anchor is anchored to the ventricular tissue, the lateral surface of each wing of the pair remains in contact with its respective leaflet during diastole.

For some applications, the leaflet support includes a flexible frame, and each wing of the pair is fixed upon the frame.

For some applications, the frame is elastically deformable.

For some applications, the frame is configured to bias the wings of the pair away from each other.

For some applications, the frame is sufficiently flexible that the wings of the pair can be pushed into contact with each other by a total converging force which is less than 30 g. For example, the wings may be pushed towards each other by a first force applied to a first wing and a second force applied to a second wing, such that the total converging force (i.e., the sum of the first force and the second force), is less than 30 g. For some applications, the frame is sufficiently flexible that the wings of the pair can be pushed into contact with each other by a total converging force of 0.1-30 g. For some applications, the frame is sufficiently flexible that the wings of the pair can be pushed into contact with each other by a total converging force of 1-30 g. For some applications, the frame is sufficiently flexible that the wings of the pair can be pushed into contact with each other by a total converging force of less than 20 g. For some applications, the frame is sufficiently flexible that the wings of the pair can be pushed into contact with each other by a total converging force of 0.1-20 g. For some applications, the frame is sufficiently flexible that the wings of the pair can be pushed into contact with each other by a total converging force of 1-20 g. For some applications, the frame is sufficiently flexible that the wings of the pair can be pushed into contact with each other by a total converging force of less than 10 g. For some applications, the frame is sufficiently flexible that the wings of the pair can be pushed into contact with each other by a total converging force of 0.1-10 g. For some applications, the frame is sufficiently flexible that the wings of the pair can be pushed into contact with each other by a total converging force of 1-10 g. For some applications, the frame is sufficiently flexible that the wings of the pair can be pushed into contact with each other by a total converging force of less than 5 g. For some applications, the frame is sufficiently flexible that the wings of the pair can be pushed into contact with each other by a total converging force of 0.1-5 g. For some applications, the frame is sufficiently flexible that the wings of the pair can be pushed into contact with each other by a total converging force of 1-5 g.

For some applications, each wing of the pair includes a fabric. For some applications, the fabric includes a polyester fabric.

For some applications, each wing of the pair includes an integrin-binding ligand.

For some applications, each wing of the pair includes fibronectin.

For some applications, each wing of the pair includes vitronectin.

For some applications, each wing of the pair includes collagen.

For some applications, each wing of the pair includes laminin.

There is further provided, in accordance with some applications, a method for use with a valve of a heart of a subject, the valve being upstream of a ventricle of the heart, and the method including transluminally advancing, to the heart, a leaflet support. For some applications, the leaflet support includes a tissue anchor and a pair of wings, each wing of the pair defining a medial surface and a lateral surface. The method further includes anchoring the tissue anchor to ventricular tissue of the ventricle.

For some applications, the method includes positioning the leaflet support in the heart, such that the tissue anchor flexibly supports the pair of wings at the valve. For some applications, this includes positioning the leaflet support such that the lateral surface of each wing of the pair faces a respective leaflet of the valve and the medial surfaces of the wings of the pair face each other. For some applications, the leaflet support is positioned such that, during systole, the lateral surface of each wing is in contact with the respective leaflet, and the medial surfaces of the wings move into contact with each other, thereby obstructing retrograde blood flow therebetween, and during diastole, the medial surfaces of the wings move out of contact with each other, thereby facilitating antegrade blood flow therebetween.

For some applications, positioning the leaflet support in the heart such that the tissue anchor flexibly supports the pair of wings at the valve, includes positioning the leaflet support in the heart such that each wing of the pair extends from a first commissure of the valve to a second commissure of the valve.

For some applications, positioning the leaflet support in the heart includes positioning the leaflet support in the heart such that, during diastole, the lateral surface of each wing of the pair remains in contact with the respective leaflet, thereby obstructing antegrade blood flow between each lateral surface and the respective leaflet, and the medial surfaces of the pair of wings move away from each other to define a medial passage therebetween, thereby facilitating antegrade blood flow through the medial passage.

For some applications, positioning the leaflet support in the heart includes coupling the pair of wings to the tissue anchor subsequently to anchoring the tissue anchor to the ventricular tissue.

For some applications, the valve is a tricuspid valve of the heart, and positioning the leaflet support in the heart such that the tissue anchor flexibly supports the pair of wings at the valve, includes positioning the leaflet support in the heart such that the tissue anchor flexibly supports the pair of wings at the tricuspid valve.

For some applications, the valve is a mitral valve of the heart, and positioning the leaflet support in the heart such that the tissue anchor flexibly supports the pair of wings at the valve, includes positioning the leaflet support in the heart such that the tissue anchor flexibly supports the pair of wings at the mitral valve.

For some applications, anchoring the tissue anchor to ventricular tissue of the ventricle includes anchoring the tissue anchor to a papillary muscle of the ventricle.

For some applications, the method includes affixing the lateral surface of at least one wing of the pair to the respective leaflet of the valve.

For some applications, affixing the lateral surface of the at least one wing to the respective leaflet of the valve includes, using a staple to staple the lateral surface of the at least one wing to the respective leaflet of the valve.

For some applications, affixing the lateral surface of the at least one wing to the respective leaflet of the valve includes, using an adhesive to adhere the lateral surface of the at least one wing to the respective leaflet of the valve.

The above method(s) can be performed on a living animal or on a simulation, such as on a cadaver, cadaver heart, simulator (e.g., with the body parts, heart, tissue, etc. being simulated), etc.

There is further provided, in accordance with some applications, a system and/or an apparatus for use with a valve of a heart of a subject, the valve having a first leaflet and a second leaflet, and the system and/or apparatus including a leaflet support. For some applications, the leaflet support includes a coaptation element having a first leaflet-contacting surface and a second leaflet-contacting surface, and a fixator fixedly coupled to the coaptation element.

For some applications, the fixator is implantable at a site behind a downstream surface of the first leaflet (e.g., underneath or on the ventricular side of the leaflet), and is configured to, while disposed behind the downstream surface of the first leaflet, passively fill with blood and support the coaptation element between the first leaflet and the second leaflet such that, during systole of the heart, the second leaflet coapts with the coaptation element.

For some applications, the leaflet support has a delivery state in which the leaflet support is collapsed for transluminal advancement to the valve.

For some applications, the fixator includes a shape-memory structure.

For some applications, the fixator includes an absorbent material, and the absorbent material is configured to passively fill with blood.

For some applications, the fixator is configured such that, while the leaflet support remains implanted at the site, the passive filling of the absorbent material with blood secures the fixator at the site.

For some applications, the fixator is configured such that, while the leaflet support remains implanted at the site, the passive filling of the absorbent material with blood pushes the first leaflet toward the second leaflet.

For some applications, the fixator is configured such that, while the leaflet support remains implanted at the site, the passive filling of the absorbent material with blood presses the first leaflet against the first leaflet-contacting surface of the coaptation element.

For some applications, the leaflet support includes a flexible frame, and the coaptation element is fixed upon the frame.

For some applications, the frame is elastically deformable.

For some applications, the frame is biased such that while the fixator is implanted at the site and supports the coaptation element between the first leaflet and the second leaflet, the frame maintains the first leaflet-contacting surface in contact with the first leaflet.

For some applications, the coaptation element includes a fabric. For some applications, the fabric includes a polyester fabric.

For some applications, the coaptation element includes an integrin-binding ligand.

For some applications, the coaptation element includes fibronectin.

For some applications, the coaptation element includes vitronectin.

For some applications, the coaptation element includes collagen.

For some applications, the coaptation element includes laminin.

There is further provided, in accordance with some applications, a method for use with a valve of a heart of a subject, the valve having a first leaflet and a second leaflet, and the method including transluminally advancing, to the valve, a leaflet support. For some applications, the leaflet support includes a coaptation element having a first leaflet-contacting surface and a second leaflet-contacting surface and a fixator fixedly coupled to the coaptation element.

The method can further include subsequently placing the fixator at a site behind a downstream surface of the first leaflet, such that the fixator expands from a contracted state to an expanded state, passively fills with blood, and supports the coaptation element between the first leaflet and the second leaflet such that, during systole of the heart, the second leaflet coapts with the coaptation element.

For some applications, placing the fixator at the site behind the downstream surface of the first leaflet such that the fixator supports the coaptation element between the first leaflet and the second leaflet, includes placing the fixator at the site such that the coaptation element extends from a first commissure of the valve to a second commissure of the valve.

For some applications, placing the fixator at the site behind the downstream surface of the first leaflet such that the fixator supports the coaptation element between the first leaflet and the second leaflet, includes placing the fixator at the site such that the first leaflet-contacting surface maintains constant contact with the first leaflet.

For some applications, placing the fixator at the site includes placing the fixator between chordae tendineae of the heart.

For some applications, placing the fixator at the site includes placing the fixator behind chordae tendineae that are connected to the first leaflet.

For some applications, placing the fixator at the site includes placing the fixator in a subannular groove of the valve.

For some applications, placing the fixator at the site behind the downstream surface of the first leaflet such that the fixator expands from the contracted state to the expanded state, includes placing the fixator at the site such that the fixator presses the first leaflet against the coaptation element.

For some applications, placing the fixator at the site behind the downstream surface of the first leaflet such that the fixator expands from the contracted state to the expanded state, includes placing the fixator at the site such that the fixator pushes the first leaflet toward the second leaflet.

For some applications, placing the fixator at the site such that the fixator passively fills with blood, includes placing the fixator at the site such that the passive filling of the fixator with blood secures the fixator to the site.

For some applications, the fixator includes an absorbent material, and placing the fixator at the site includes placing the fixator at the site such that the absorbent material passively fills with blood.

For some applications, the valve is a mitral valve of the heart, and transluminally advancing the leaflet support to the valve includes transluminally advancing the leaflet support to the mitral valve.

For some applications, the first leaflet is a posterior leaflet of the mitral valve, the second leaflet is an anterior leaflet of the mitral valve, the site is a site behind a downstream surface of the posterior leaflet, and placing the fixator at the site includes placing the fixator at the site behind the downstream surface of the posterior leaflet.

The above method(s) can be performed on a living animal or on a simulation, such as on a cadaver, cadaver heart, simulator (e.g., with the body parts, heart, tissue, etc. being simulated), etc.

There is further provided, in accordance with some applications, a system or and/or an apparatus for use with a valve of a heart of a subject, the valve having a first leaflet and a second leaflet, and the system and/or apparatus including a leaflet support. For some applications, the leaflet support includes a coaptation element having a first leaflet-contacting surface and a second leaflet-contacting surface, and a fixator coupled to the coaptation element.

For some applications, the fixator may be implantable at a site downstream of the valve, e.g., within a ventricle, and configured to support the coaptation element between the first leaflet and the second leaflet such that, during systole of the heart, the first leaflet is in contact with the first leaflet-contacting surface, and the second leaflet coapts with the second leaflet-contacting surface.

For some applications, during diastole the first leaflet remains in contact with the first leaflet-contacting surface, while the second leaflet moves away from the second leaflet-contacting surface, thereby facilitating antegrade blood flow through the valve.

For some applications, the fixator may be implantable at a site behind a downstream surface of the first leaflet, and configured to, while disposed behind the downstream surface of the first leaflet, passively fill with blood.

For some applications, the fixator may be a tissue anchor, anchorable to ventricular tissue of a ventricle that is downstream of the valve.

The present invention will be more fully understood from the following detailed description of applications thereof, taken together with the drawings, in which:

### BRIEF DESCRIPTION OF THE DRAWINGS

FIGS. 1-2 are schematic illustrations showing a leaflet support, and the use thereof to facilitate coaptation of leaflets of a valve of a heart of a subject, in accordance with some applications; and

FIGS. 3-4 are schematic illustrations showing a leaflet support and the use thereof to facilitate coaptation of leaflets of the valve, in accordance with some applications.

### DETAILED DESCRIPTION

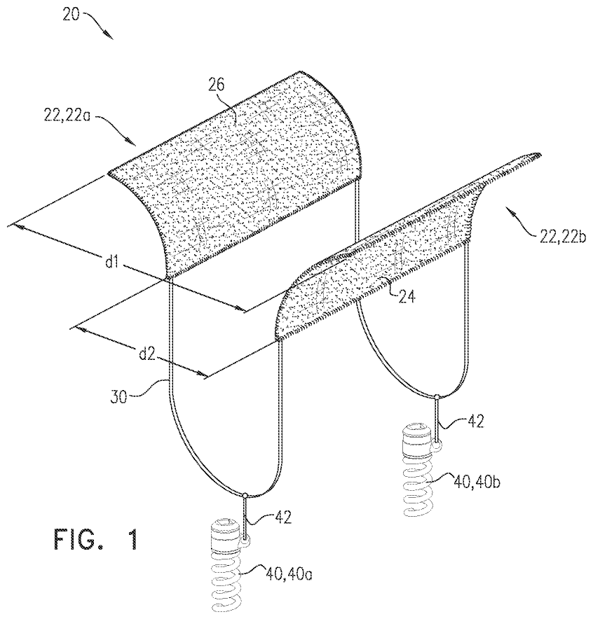

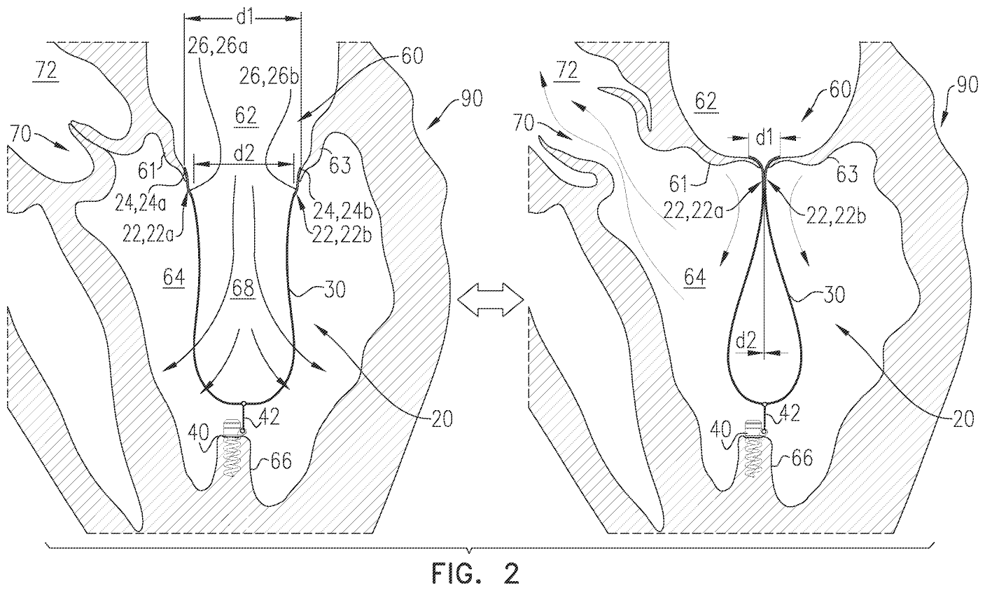

Reference is made to FIGS. 1-2, which are schematic illustrations showing a leaflet support 20, and the use thereof to facilitate coaptation of leaflets 61, 63 of a valve 60 of a heart 90 of a subject, in accordance with some applications.

Leaflet support 20 comprises a pair of wings 22 (e.g., a first wing 22a and a second wing 22b), which are coupled to each other such that, when leaflet support is implanted in heart 90, (i) during systole, the medial surfaces of the wings of the pair move into contact with each other, thereby obstructing retrograde blood flow through the valve, and (ii) during diastole, the medial surfaces of the wings move out of contact with each other, thereby facilitating antegrade blood flow through the valve. For example, and as shown as in FIG. 1, wings 22 can be coupled to each other via a flexible frame 30, such as a wire.

For some applications, and as shown, wings 22 are positioned such that each wing has a medial surface 26 facing the medial surface of the other wing, as well as a lateral surface 24 facing away from the other wing. For some such applications, and as shown, wings 22 can be shaped as contoured sheets that curve apart from each other as they extend (e.g., as they extend upstream) along a respective contour of leaflets 61, 63. For example, an upstream inter-wingspan d1 between upstream ends of the wings, can be greater than a downstream inter-wingspan d2 between downstream ends of the wings.

For some applications, and as shown, support 20 further comprises a tissue anchor 40 (e.g., a pair of tissue anchors 40a, 40b). Often for such applications, support 20 is coupled to the anchor 40 such that the anchor flexibly supports wings 22 at valve 60. For some applications (not shown), anchors 40 are coupled directly to frame 30 of support 20. For some applications, and as shown, a rod 42 is used to couple wings 22 to anchors 40. For some applications (not shown), frame 30 comprises a hinge (e.g., by a hinge coupling the frame to anchor 40 and/or to rod 42).

For some applications, and as shown, wings 22 are fixed upon flexible frame (e.g., wire) 30, such that the frame serves as a scaffolding for the wings. Wings 22 can be comprised of a material that has greater flexibility than the frame, and therefore the wings can generally assume a shape defined by the frame. For example, and as shown, frame 30 can be secured along one or more edges of wings 22. Alternatively or in addition, it may be desirable for frame 30 to support wings 22 between the edges.

For some applications, frame 30 is elastically deformable (e.g., comprising an elastic, superelastic, or shape-memory material such as Nitinol), and is biased to maintain wings 22 apart in the absence of force applied thereto. For some such applications, frame 30 is sufficiently flexible that wings 22 can be pushed into contact with each other by forces experienced during systole (e.g., forces applied by blood and/or leaflets 61, 63). For example, frame 30 can be sufficiently flexible that wings 22 can be pushed into contact with each other (e.g., during systole) by a total medial pushing (i.e., converging) force of less than 30 g (e.g., less than 20 g, e.g., less than 10 g, e.g., less than 5 g) and/or greater than 0.1 g (e.g., greater than 1 g), e.g., 0.1-30 g (e.g., 0.1-20 g, 0.1-10 g, e.g., 0.1-5 g) or 1-30 g (e.g., 1-20 g, e.g., 1-10 g, e.g., 1-5 g). It is hypothesized that such a configuration facilitates maintenance of contact between leaflets 61, 63 and wings 22 while the heart cycles from diastole to systole.

FIG. 2 shows support 20 having been implanted into heart 90 (e.g., after the support has been transluminally advanced in a delivery state in which frame 30 and/or wings 22 are compressed, and after the support has been subsequently expanded in the heart). For some applications, and as shown, anchors 40 are anchored to tissue of left ventricle 64 (e.g., to papillary muscle 66 thereof), downstream of mitral valve 60. Although support 20 is described herein as being used to facilitate coaptation of the leaflets (e.g., an anterior leaflet 61 and a posterior leaflet 63) of mitral valve 60, this is not meant to exclude use of support 20 at other valves of heart 90, mutatis mutandis. Thus, although one pair of wings 22 can be sufficient to facilitate coaptation of leaflets 61, 63 of mitral valve 60, it may be desirable to position additional pairs of wings 22 into other valves. For example, at the tricuspid valve, it may be desirable to utilize two or three pairs of wings 22 (e.g., two or three leaflet supports 20, or a leaflet support that itself comprises two or three pairs of wings).

For some applications, wings 22 and leaflets 61, 63 move in tandem (e.g., toward each other and away from each other) while the heart cycles between diastole (left frame of FIG. 2) and systole (right frame of FIG. 2). It is to be noted that, in contrast to leaflets of a prosthetic heart valve, in which a first portion of one leaflet moves in relation to a first portion of another leaflet while a second portion of each leaflet remains stationary (e.g., the second portion remains affixed to a valve frame), in leaflet support 20 wings 22 typically move in their entirety toward and away from each other. For example, both upstream inter-wingspan d1 and downstream inter-wingspan d2 are increased during diastole and reduced during systole.

Whereas leaflets of a prosthetic heart valve typically contact each other at the edges of the leaflets, for some applications, medial surfaces 26 of wings 22 contact each other on faces of the wings, but not at edges of the wings.

It is typically desirable that wings 22 facilitate antegrade (downstream) blood flow (e.g., from left atrium 62 to left ventricle 64, through mitral valve 60), during diastole (indicated by arrows in left frame of FIG. 2). During diastole, blood typically flows downstream through a medial passage 68 between medial surfaces 26 (e.g., between medial surfaces 26a, 26b of the respective wings).

For some applications, each wing 22 (e.g., lateral surface 24 thereof) remains in contact with its respective leaflet 61, 63 during diastole. For some such applications, elasticity of the frame is such that, while wings 22 are positioned between leaflets 61, 63, the wings apply mild a lateral pushing force against the leaflets, thereby maintaining the wings (e.g., lateral surfaces 24 thereof) in contact with the leaflets. It is hypothesized that wings 22 contacting the leaflets during diastole serves to reduce obstruction of antegrade blood flow by the wings.

For some applications, maintenance of contact between lateral surfaces 24 and respective leaflets during diastole is facilitated by affixing lateral surfaces 24 to leaflets 61, 63. For example, a staple can be used to staple each wing to a leaflet. Alternatively or in addition, an adhesive can be used to adhere the lateral surfaces to the leaflets. Alternatively still, or in addition, lateral surfaces 24 can comprise a material that encourages growth of tissue of leaflets 61, 63 into the lateral surfaces. For example, lateral surfaces 24 can comprise a fabric such as a polyester (e.g., polyethylene terephthalate) fabric, and/or an integrin-binding ligand (e.g., fibronectin, vitronectin, collagen and/or laminin).

For some applications, wings 22 are dimensioned to avoid obstruction of antegrade blood flow during diastole. For example, each entire wing 22 can be disposed flat against its respective leaflet 61, 63 (e.g., lateral surface comprises an entire area of wing 22) while the support is anchored to the ventricular tissue. In this way, wings 22 may not be anticipated to obstruct antegrade blood flow significantly more than do the leaflets.

For some applications, during systole, coaptation between medial surfaces 26 of wings 22 closes support 20 to blood flow therethrough. It is hypothesized that contact between (i) lateral surface 24a and anterior leaflet 61, (ii) lateral surface 24b and posterior leaflet 63, and (iii) respective medial surfaces 26a and 26b, each alone and in tandem, obstructs retrograde blood flow through mitral valve 60 during systole. It is further hypothesized that obstructing retrograde blood flow through mitral valve 60 during systole may facilitate antegrade blood flow through aortic valve 70 into aorta 72 (as indicated by arrows in right frame of FIG. 2b).

For some applications, wings 22 comprise material that is impermeable to blood. For some applications, wings 22 comprise pericardial tissue. It is hypothesized that such materials may facilitate obstruction of retrograde blood flow through mitral valve 60 during systole.

For some applications, support 20 is dimensioned such that each wing 22 extends from a first commissure to a second commissure of the valve (e.g., from an anterior commissure of mitral valve 60 to a posterior commissure of the mitral valve). It is hypothesized that each wing of support 20 extending from commissure to the commissure facilitates obstruction of retrograde blood flow through by the support more effectively than if wings 22 were to extend only partly between commissures.

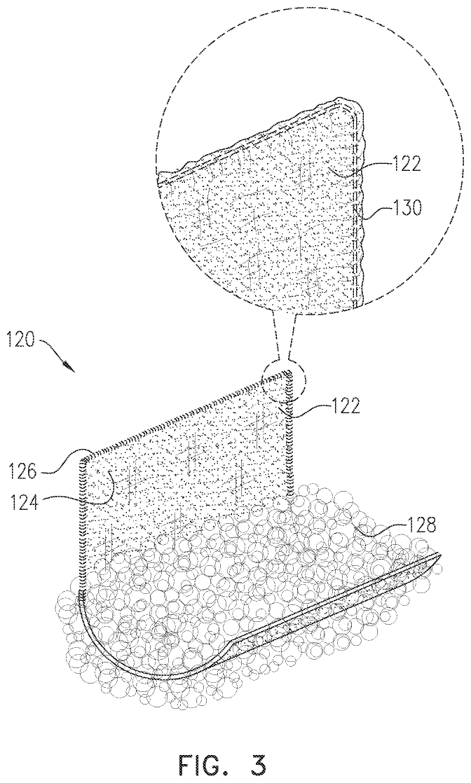

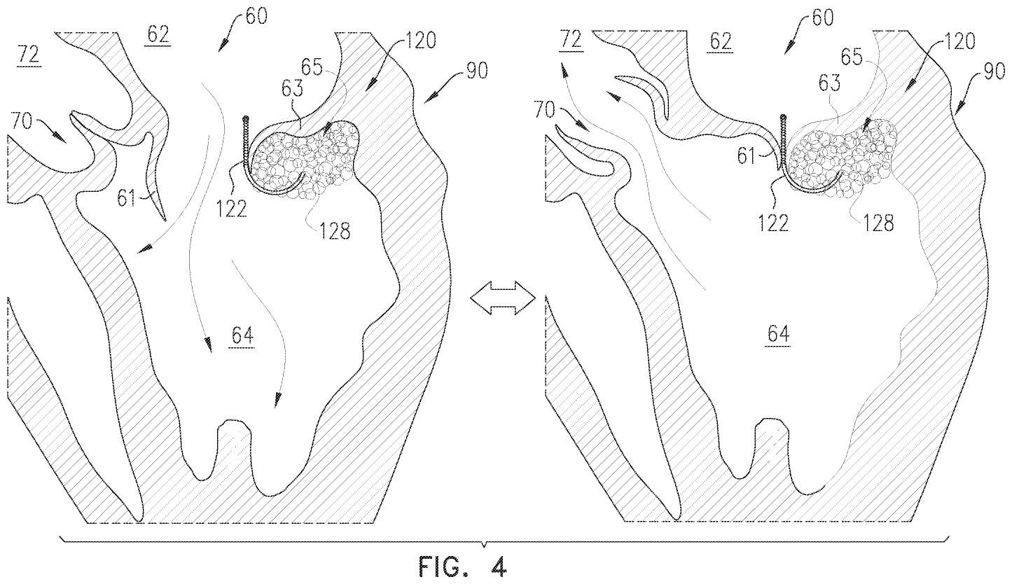

Reference is made to FIGS. 3-4, which are schematic illustrations showing a leaflet support 120 and the use thereof to facilitate coaptation of leaflets 61, 63 of valve 60, in accordance with some applications.

Similar to support 20 described hereinabove, support 120 is shown being used to facilitate coaptation of mitral valve 60, yet this description is not meant to exclude use of support 120 to facilitate coaptation of other valves of the heart, mutatis mutandis.

As shown in FIG. 3, support 120 comprises a fixator 128 that is coupled to a coaptation element 122. Support 120 is shown in an expanded working state. However, support 120 can be transluminally delivered to valve 60 in a delivery state in which both coaptation element 122 and fixator 128 are compressed.

Support 120 shares some features with support 20 described hereinabove. Particularly, coaptation element 122 shares some commonalities with wing 22 (However, in contrast to support 20 which comprises a pair of wings, support 120 comprises a single coaptation element 122). For instance, coaptation element 122 can comprise a frame (e.g., comprising a shape-memory material) 130 which serves as a scaffolding that defines a shape of the coaptation element. For some such applications, and as shown in FIG. 4, frame 130 is biased to maintain first leaflet-contacting surface 124 in contact (e.g., in constant contact) with posterior leaflet 63.

For some applications, and similarly to as described hereinabove in reference to lateral surfaces 24 of wings 22 of support 20, first leaflet-contacting surface 124 comprises material that encourages growth of tissue (e.g., tissue of posterior leaflet 63) positioned into the first leaflet-contacting surface. For example, first leaflet-contacting surface 124 can comprise a fabric such as a polyester (e.g., polyethylene terephthalate) fabric, and/or an integrin-binding ligand (e.g., fibronectin, vitronectin, collagen and/or laminin).

As shown in FIG. 4, coaptation element 122 can be deployed such that a first leaflet-contacting surface 124 faces posterior leaflet 63, and a second leaflet-contacting surface 126 faces anterior leaflet 61. As shown in the right pane of FIG. 4, anterior leaflet 61 can coapt with coaptation element 122 (e.g., with second leaflet-contacting surface 126 thereof) during systole, obstructing retrograde blood flow through valve 60. During systole (left pane of FIG. 4), anterior leaflet 61 can deflect, relative to second leaflet-contacting surface 126, facilitating antegrade blood flow through valve 60.

As described hereinabove in reference to wings 22 of support 20, coaptation element 122 can extend from the anterior commissure of mitral valve 60 to the posterior commissure of the mitral valve. It is hypothesized that coaptation element 122 extending from the anterior commissure to the posterior commissure impedes retrograde blood flow through mitral valve 60 during systole, more effectively than would support 120 if the coaptation element were to extend only partly between commissures.

FIG. 4 shows fixator 128 having been placed (e.g., implanted) at an implantation site behind (e.g., downstream of) a downstream surface 65 of posterior leaflet 63 of mitral valve 60. For some applications, fixator 128 is allowed to expand into the working state, while disposed at the implantation site, such that the fixator contacts posterior leaflet 63 (e.g., downstream surface 65 thereof) and other ventricular tissue of heart 90 (e.g., chordae tendineae thereof). For some applications, fixator 128 is positioned behind posterior leaflet 63 leaflet and chordae tendineae that are connected to the posterior leaflet, e.g., subannularly, such as in the subannular groove. Fixator 128 expands after its implantation, such that the volume it eventually occupies (e.g., its bulk) anchor it in place. It is hypothesized that contact between fixator 128, posterior leaflet 63 and the other ventricular tissue may obviate use of a tissue anchor to anchor leaflet support 120.

For some applications, e.g., for applications in which fixator 128 is disposed downstream of mitral valve 60, fixator 128 can be substituted by a tissue anchor, e.g., tissue anchors 40, sharing the same features as described hereinabove with respect to FIGS. 1 and 2.

For some applications, the expansion of fixator 128 can press posterior leaflet 63 against coaptation element 122 (e.g., against first leaflet-contacting surface 124 thereof), and/or can push the posterior leaflet toward anterior leaflet 61. It is hypothesized that, for some applications, this may facilitate coaptation of anterior leaflet 61 with coaptation element 122 during systole.

For some applications, fixator 128 passively fills with blood upon implantation at the implantation site. For some such applications, fixator 128 filling with blood facilitates securing fixator 128 to the implantation site (e.g., by increasing a volume of the fixator, thereby increasing contact between the fixator and the implantation site). For some such applications, fixator 128 comprises an absorbent material. For some such applications, the absorbent material filling with blood facilitates clotting of the blood within fixator 128, and/or tissue growth on the fixator. For example, clotting of the blood within fixator 128, while the fixator contacts the posterior leaflet 63, may serve to fixate the posterior leaflet.

Alternatively or in addition to comprising absorbent material, fixator 128 can further comprise a self-expanding structure. For example, nonabsorbent fixator 128 can comprise a shape-memory spring or other structure.

The present invention is not limited to the examples that have been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the techniques, methods, operations, steps, etc. described or suggested herein can be performed on a living animal or on a non-living simulation, such as on a cadaver, cadaver heart, simulator (e.g., with the body parts, tissue, etc. being simulated), etc.

1. A system for use with a valve of a heart of a subject, the heart cycling between systole and diastole, and the system comprising a leaflet support, the leaflet support comprising:
a tissue anchor, anchorable to ventricular tissue of a ventricle that is downstream of the valve; and
a pair of wings, each wing of the pair defining:
a medial surface, such that the medial surface of one wing of the pair faces the medial surface of the other wing of the pair, and
a lateral surface,
wherein the pair of wings is coupled to the tissue anchor such that, when the tissue anchor is anchored to the ventricular tissue, the tissue anchor flexibly supports the pair of wings at the valve, with the lateral surface of each wing of the pair facing a respective leaflet of the valve such that:
during systole:
the lateral surface of each wing of the pair is in contact with the respective leaflet, and
the medial surfaces of the wings of the pair move into contact with each other, thereby obstructing retrograde blood flow through the valve, and
during diastole, the medial surfaces of the wings move out of contact with each other, thereby facilitating antegrade blood flow through the valve.

2. The system according to claim 1, wherein the lateral surface comprises an entire area of the wing.

3. The system according to claim 1, wherein the leaflet support has a delivery state in which the leaflet support is configured to be transluminally advanceable to the valve.

4. The system according to claim 1, wherein the pair of wings is a first pair of wings, and wherein the leaflet support further comprises a second pair of wings.

5. The system according to claim 4, wherein the leaflet support further comprises a third pair of wings.

6. The system according to claim 1, wherein the pair of wings is configured such that, when the tissue anchor is anchored to the ventricular tissue, each wing of the pair remains in contact with its respective leaflet during diastole.

7. The system according to claim 6, wherein the pair of wings is configured such that, when the tissue anchor is anchored to the ventricular tissue, the lateral surface of each wing of the pair remains in contact with its respective leaflet during diastole.

8. The system according to claim 1, wherein the leaflet support further comprises a flexible frame, and each wing of the pair is fixed upon the frame.

9. The system according to claim 8, wherein the frame is elastically deformable.

10. The system according to claim 9, wherein the frame is configured to bias the wings of the pair away from each other.

11. The system according to claim 10, wherein the frame is sufficiently flexible that the wings of the pair can be pushed into contact with each other by a total converging force of less than 30 g.

12. The system according to claim 11, wherein the frame is sufficiently flexible that the wings of the pair can be pushed into contact with each other by a total converging force of less than 20 g.

13. The system according to claim 12, wherein the frame is sufficiently flexible that the wings of the pair can be pushed into contact with each other by a total converging force of less than 10 g.

14. The system according to claim 13, wherein the frame is sufficiently flexible that the wings of the pair can be pushed into contact with each other by a total converging force of 0.1-10 g.

15. The system according to claim 14, wherein the frame is sufficiently flexible that the wings of the pair can be pushed into contact with each other by a total converging force of 1-10 g.

16. The system according to claim 13, wherein the frame is sufficiently flexible that the wings of the pair can be pushed into contact with each other by a total converging force of less than 5 g.

17. The system according to claim 1, wherein each wing of the pair comprises an integrin-binding ligand.

18. The system according to claim 17, wherein each wing of the pair comprises at least one of fibronectin, vitronectin, collagen, and laminin.

19. The system according to claim 1, wherein each wing of the pair comprises a material that is impermeable to blood.

20. The system according to claim 1, wherein each wing of the pair comprises pericardial tissue.

21. The system according to claim 1, wherein each wing of the pair comprises a fabric.
